(19) United States
(12) Patent Application Publication
(10) Pub. No.: US 2018/0317797 A1
(43) Pub. Date: Nov. 8, 2018

(54) CONVERTIBLE ELECTRODE PATCH

(71) Applicant: Applied Cardiac Systems, Inc., Irvine, CA (US)

(72) Inventor: Loren A. Manera, Laguna Hills, CA (US)

(73) Assignee: ACS Diagnostics, Inc., Irvine, CA (US)

(21) Appl. No.: 15/971,945

(22) Filed: May 4, 2018

Related U.S. Application Data
(60) Provisional application No. 62/502,475, filed on May 5, 2017.

(51) Int. Cl. A61B 5/0408

(52) U.S. Cl. CPC A61B 5/04085; A61B 2090/037; A61B 5/04087

(57) ABSTRACT

An electrode patch including a patch body and a plurality of electrodes attached to the patch body for sensing EKG signals. The electrode patch also includes at least one perforation on the patch body to help define multiple portions of patch body. Each portion of the patch body is attached to one of the plurality of electrodes. The electrode patch may be secured to a body of a patient in an intact configuration having each portion of the patch body connected together or in a separated configuration having at least one portion separated from the other portions of the patch body in order to accommodate different sizes and shapes of patients.

### CROSS-REFERENCE TO RELATED CASES

This application claims the benefit of U.S. Provisional Application No. 62/502,475, filed May 5, 2017, which is herein incorporated by reference in its entirety.

### FIELD OF THE DISCLOSURE

The present disclosure relates generally to an electrode patch that may be used with health monitoring device.

### BACKGROUND

In the EKG industry, the single electrode or patch is popular for patient comfort. However, at least half of the time, a single patch/electrode design cannot be used on different body types and sizes, for example between a male and a female. In that case, individual electrodes are necessary. What is needed is a convertible or adjustable electrode (patch) design that allows the medical technician or physician to have the advantages of the single electrode design, but also allows the physician to convert the electrode (patch) into as many individual electrodes as necessary.

### SUMMARY

Briefly, and in general terms, the present disclosure is directed to an electrode patch. The electrode patch includes a patch body and a plurality of electrodes attached to the patch body for sensing EKG signals. The electrode patch also includes at least one perforation on the patch body to help define multiple portions of patch body. Each portion of the patch body is attached to one of the plurality of electrodes. In one embodiment, the electrode patch includes a negative electrode attached to a first portion of the patch body, a ground electrode attached to a second portion of the patch body, and a positive electrode attached to a third portion of the patch body. The electrode patch may be secured to a body of a patient in an intact configuration having each portion of the patch body connected together or in a separated configuration having at least one portion separated from the other portions of the patch body in order to accommodate different sizes and shapes of patients.

In one embodiment, the patch body of the electrode patch includes an adhesive material for securing the electrode patch to a body of a patient. There may also be an electrode conductive gel covering a bottom portion of the plurality of electrodes on the electrode patch.

In one embodiment, the patch body of the electrode patch includes a first portion, a second portion and a third portion, and each portion of the patch body is separated by a perforation. There may be additional portions of the patch body, with each portion separated by a perforation. The perforations allows a medical technician to easily separate the portions of the patch body from one another in order to place the portions and associated electrodes at different distances from one another depending on the body of the patient. The electrode patch may include an intact configuration having each portion of the patch body connected together. Also, the electrode patch includes a separated configuration having at least one portion separated from the other portions of the patch body. It has been contemplated that the electrode patch can be converted from the intact configuration into the separated configuration by cutting the patch body or separating the electrodes by any other method.

The present disclosure also is directed to a cardiac monitoring system for acquiring ECG signals. In one embodiment, the system includes a cardiac monitoring device and a plurality of lead wires attached to the cardiac monitoring device. There is also an electrode patch including a patch body and a plurality of electrodes attached to the patch body for sensing ECG signals. The patch body includes at least one perforation to define multiple portions of patch body and each portion of the patch body is attached to one of the plurality of electrodes. Each of the plurality of electrodes is attached to one of the plurality of lead wires and in communication with the cardiac monitoring device.

Furthermore, the present disclosure is directed to a method for acquiring ECG signals. The method includes attaching an electrode patch to at least one lead wire of a cardiac monitor. As described above, the electrode patch may include a patch body and a plurality of electrodes attached to the patch body for sensing ECG signals. The patch body includes at least one perforation to define multiple portions of patch body and each portion of the patch body is attached to one of the plurality of electrodes. Each of the plurality of electrodes is attached to one lead wire and is in communication with the cardiac monitoring device. The method also includes attaching the electrode patch to a body of a patient. The electrode patch may remain in an intact configuration having each portion of the patch body connected together. In another embodiment, the method includes tearing the patch body along the at least one perforation to place the electrode patch in a separated configuration. Once in the separated configuration, the multiple portions of the patch body may be placed in desired locations on the body of the patient without being constrained by the size and shape of the intact patch body.

These and other features and advantages will become further apparent from the detailed description and accompanying figures that follow. In the figures and description, numerals indicate the various features of the disclosure, like numerals referring to like features throughout both the drawings and the description.

### BRIEF DESCRIPTION OF THE DRAWINGS

The teachings described herein are further described in terms of exemplary embodiments. These exemplary embodiments are described in detail with reference to the drawings. These embodiments are non-limiting exemplary embodiments.

FIGS. 1A depicts a top side of one embodiment of an electrode patch showing perforations at various locations on the patch. The sites on the patch may be color coded, for example, white, black, and red.

FIG. 1B depicts an embodiment of the electrode patch detached to accommodate a location for a negative electrode.

FIG. 1C depicts an embodiment of the electrode patch detached to accommodate a location for a positive electrode.

FIG. 1D depicts an embodiment of the electrode patch completely detached at each perforation spot to accommodate different patient body shapes and sizes.

FIG. 2 depicts a bottom side of the electrode patch shown in FIG. 1. An electrode conductive gel may be used on the bottom side of the patch to create electrical skin contact.

FIG. 3 depicts three leads attached to one embodiment of an electrode patch and the leads are also attached to a cardiac monitoring device. The patch is attached to the chest of a patient.

FIG. 4 depicts a completely detached embodiment of the electrode patch attached to a cardiac monitoring device with the lead wires looped under the adhesive material available after the patch is detached.

### DETAILED DESCRIPTION OF THE PREFERRED EMBODIMENTS

In electrocardiography, the word “lead” may refer to either the electrodes attached to the patient, or, properly, to the voltage or signal measured between two electrodes. To avoid confusion, “channel” is used to describe the voltage, signal or view from a positive electrode to a negative electrode across the heart.

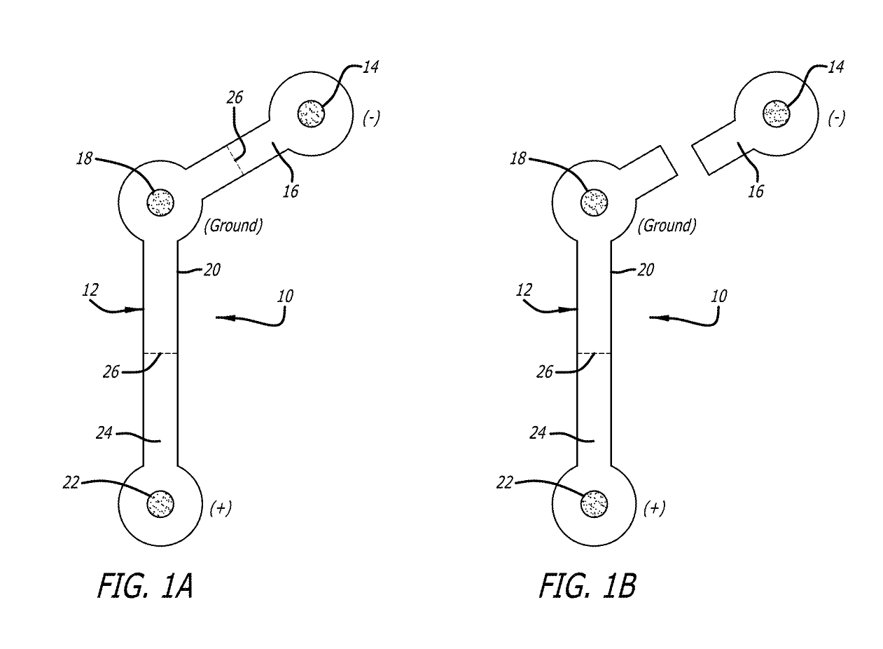

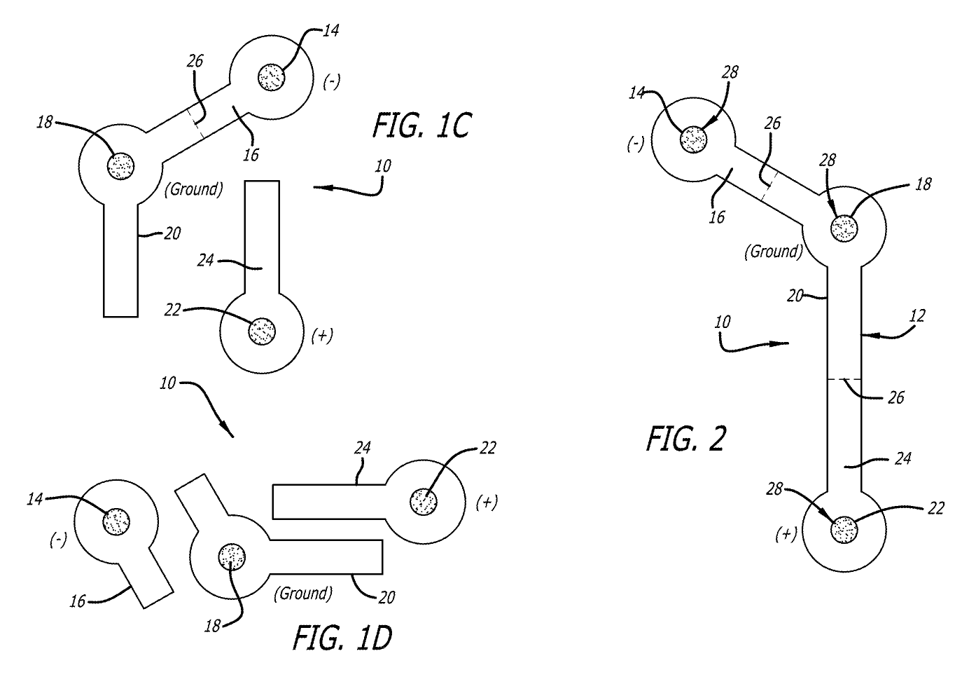

Referring now to FIG. 1A, an electrode patch 10 is shown having a patch body 12. The patch body 12 can be in various shapes and sizes. There are electrodes connected to the patch body. In the embodiment shown, there is a negative electrode 14 on a first portion 16 of the patch body, a ground electrode 18 on a second portion 20 of the patch body, and a positive electrode 22 on a third portion of the patch body. Perforations 26 are also on the patch body 12 in order to tear and remove one portion of the patch body. The perforations 26 may be located anywhere in between the individual electrodes. In one embodiment the perforations set the boundaries for the first, second and third portions of the patch body. In other embodiments, the electrode patch may be separated into distinct portions by other methods including cutting or tearing the patch body itself Also, in other embodiments, additional electrodes may be included on the patch body and the number of perforations may increase depending on the number of electrodes. As shown in FIG. 1A, the electrode patch 10 is intact.

In use, the electrode patch 10 may be used with a cardiac monitoring device and placed on a patient for observation. One embodiment of the electrode patch 10 is designed to maximize patient comfort while improving patient compliance. One embodiment of the electrode patch is a combination of an EKG patch (multiple electrodes housed in one shape or body) and when necessary, a separated or “spider” design can be converted into multiple individual electrodes.

An embodiment of the electrode patch 10 detached to accommodate a location for a negative electrode 14 is shown in FIG. 1B. As shown, one perforation 26 has been torn separating the first portion 16 from the other portions of the electrode patch body 12. In this way, the negative electrode may be placed in a more desired location on the body of a patient that is away from the other electrodes of the patch. In this way, the electrode patch is adjustable to the size and shape of the body of the patient.

An embodiment of the electrode patch 10 detached to accommodate a location for a positive electrode 22 is shown in FIG. 1C. As shown, one perforation 26 has been torn separating the third portion 24 from the other portions of the electrode patch body 12. In this way, the positive electrode may be placed in a more desired location on the body of a patient that is away from the other electrodes of the patch. In this way, the electrode patch is adjustable to the size and shape of the body of the patient.

An embodiment of the electrode patch 10 completely detached at each perforation 26 is shown in FIG. 1D. As shown, both perforations 26 have been torn separating the first, second, and third portions 16, 20, and 24 from one another. In this way, each negative electrode may be placed in a more desired location on the body of a patient that is away from the other electrodes of the patch. In this way, the electrode patch is adjustable to the size and shape of the body of the patient and is not restricted to the size of the electrode patch when it is intact.

FIG. 2 depicts a bottom side of the electrode patch 10 shown in FIG. 1A. The bottom side will come into contact with the patient body. There may be an electrode conductive gel 28 covering the bottom side of the electrodes on the patch to create electrical skin contact. In addition, there may be an adhesive (not shown) on the bottom side of the patch body 12 in order for the patch to attach to the body of the patient.

The electrode patch 10 shown in the figures allows a medical technician or physician to have the advantages of the single electrode design, but allows the technician or physician to convert the single patch/electrode design into as many individual electrodes as necessary depending on the body-type of the patient. This allows the technician or physician to get the necessary EKG information from a single patch design as shown. As shown, one embodiment of electrode patch 10 is for two leads of EKG. However, the perforated electrode patch can be designed to provide anywhere from one EKG lead to 12 EKG leads, depending upon which EKG tests are necessary. And in any test case, if the individual one piece design does not conform to the body of the patient, the electrode patch 10 can convert to separate electrodes and can be applied wherever on the body it is necessary to get the EKG from that patient. This design insures that a patient's EKG hookup can occur regardless of the body size and or condition of the patient's body and further insure maximum patient comfort and compliance. This design of the electrode patch 10 allows for maximum use of multiple leads, whereas other patch products do not provide for multiple EKG leads vectors.

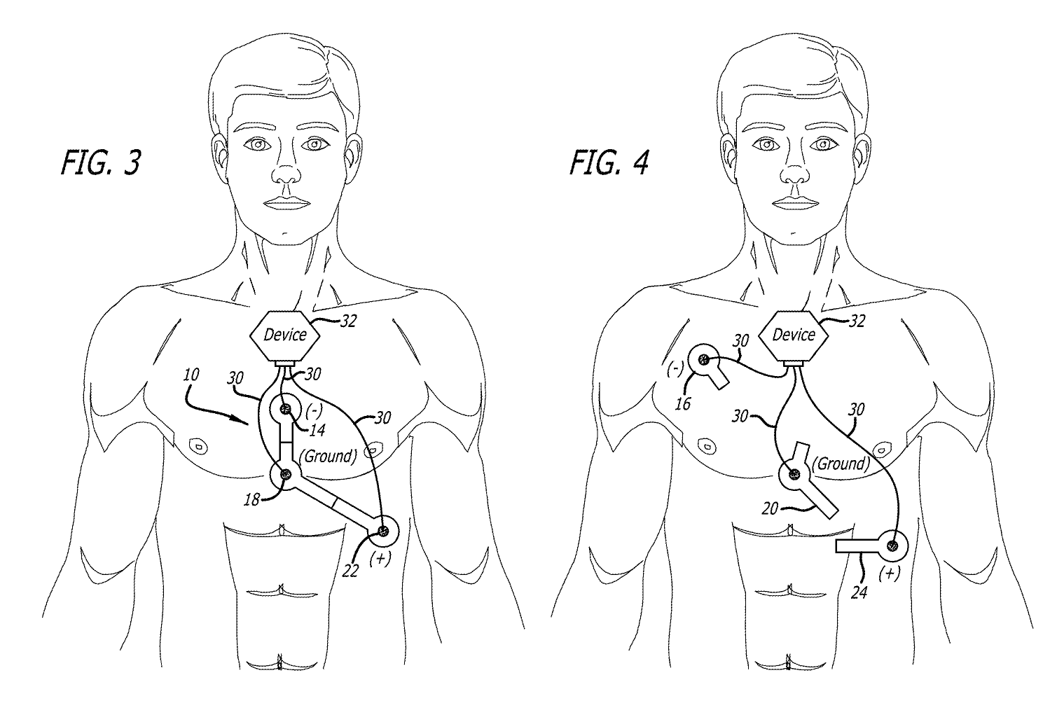

As shown in FIG. 3, the electrodes 14, 18, and 22 of the electrode patch 10 are connected to leads 30 that are connected to a cardiac monitoring device 32. As shown, the cardiac monitoring device 32 may be a portable or wearable cardiac monitoring device. In other embodiments, the electrodes of the electrode patch 10 may be connected to any cardiac monitoring device. In use, the patch 10 may be left intact as shown in FIG. 3 and secured to the body of the patient.

If necessary, the electrode patch 10 may be converted from the single patch configuration to the separated configuration, where the first, second and third portions have all been separated from one another as shown in FIG. 4. After tearing the patch body 12 at the perforations 26, the separated portions may then be secured to the body of the patient in various locations in order to collect EKG information. In one embodiment, the adhesive material between perforations on the bottom side of the patch body may be utilized to hold in place the EKG lead-wires 30. In use, the leads 30 are looped under the portions of the patch body to form stress loops as shown in FIG. 4 when the patch is secured to the body. In this way, the lead-wires remain in place.

Each of the features and teachings disclosed herein can be utilized separately or in conjunction with other features and teachings to provide an electrode patch. This detailed description is merely intended to teach a person of skill in the art further details for practicing aspects of the present teachings and is not intended to limit the scope of the present disclosure. Therefore, combinations of features disclosed above in the detailed description may not be necessary to practice the teachings in the broadest sense, and are instead taught merely to describe particularly representative examples of the present teachings.

Variations may be made to the embodiments described herein without departing from the scope of the present disclosure. All the elements described may be replaced by equivalent elements and the parts, materials, shapes and dimensions may be chosen as needed.

We claim:

1. An electrode patch, comprising:
a patch body;
a plurality of electrodes attached to the patch body for sensing EKG signals;
at least one perforation on the patch body, the at least one perforation defines multiple portions of patch body and each portion of the patch body is attached to one of the plurality of electrodes.

2. The electrode patch of claim 1, wherein the patch body includes an adhesive material for securing the electrode patch to a body of a patient.

3. The electrode patch of claim 1, further comprising an electrode conductive gel covering a bottom portion of the plurality of electrodes.

4. The electrode patch of claim 1, wherein the plurality of electrodes each include a port for attaching to a lead wire.

5. The electrode patch of claim 1, wherein the patch body includes a first portion, a second portion and a third portion, and each portion of the patch body is separated by the at least one perforation.

6. The electrode patch of claim 5, wherein the patch body includes an intact configuration having each portion of the patch body is connected together.

7. The electrode patch of claim 5, wherein the patch body includes a separated configuration having at least one portion separated from the other portions of the patch body.

8. The electrode patch of claim 5, wherein a negative electrode is attached to the first portion of the patch body, a ground electrode is attached to the second portion of the patch body, and a positive electrode is attached to the third portion of the patch body.

9. A cardiac monitoring system for acquiring ECG signals, the system comprising:
a cardiac monitoring device;
a plurality of lead wires attached to the cardiac monitoring device; and
an electrode patch including a patch body and a plurality of electrodes attached to the patch body for sensing ECG signals, the patch body including at least one perforation to define multiple portions of patch body and each portion of the patch body is attached to one of the plurality of electrodes, and each of the plurality of electrodes is attached to one of the plurality of lead wires and in communication with the cardiac monitoring device.

10. The system of claim 9, wherein the patch body includes an adhesive material for securing the electrode patch to a body of a patient.

11. The system of claim 9, further comprising an electrode conductive gel covering a bottom portion of the plurality of electrodes.

12. The system of claim 9, wherein the plurality of electrodes each include a port for attaching to the plurality of lead wires.

13. The system of claim 9, wherein the patch body includes a first portion, a second portion and a third portion, and each portion of the patch body is separated by the at least one perforation.

14. The system of claim 13, wherein the patch body includes an intact configuration having each portion of the patch body connected together.

15. The system of claim 13, wherein the patch body includes a separated configuration having at least one portion separated from the other portions of the patch body.

16. The system of claim 13, wherein a negative electrode is attached to the first portion of the patch body, a ground electrode is attached to the second portion of the patch body, and a positive electrode is attached to the third portion of the patch body.

17. A method for acquiring ECG signals, the method comprising:
attaching an electrode patch to at least one lead wire of a cardiac monitor, the electrode patch including a patch body and a plurality of electrodes attached to the patch body for sensing ECG signals, the patch body including at least one perforation to define multiple portions of patch body and each portion of the patch body is attached to one of the plurality of electrodes, and each of the plurality of electrodes is attached to at least one lead wire and in communication with the cardiac monitoring device; and
attaching the electrode patch to a body of a patient in an intact configuration having each portion of the patch body connected together or in a separated configuration having at least one portion separated from the other portions of the patch body.

18. The method of claim 17, further comprising tearing the patch body along the at least one perforation to place the electrode patch in the separated configuration.
